(19) United States
(12) Patent Application Publication
(10) Pub. No.: US 2023/0238127 A1
(43) Pub. Date: Jul. 27, 2023

(54) MEDICAL DEVICE CONTROL WITH VERIFICATION BYPASS

(71) Applicant: Cochlear Limited, Macquarie University, NSW (AU)

(72) Inventor: Kenneth OPLINGER, Macquarie University, NSW (AU)

(21) Appl. No.: 18/001,837

(22) PCT Filed: May 4, 2021
(86) PCT No.: PCT/IB2021/053723

§ 371 Date: Dec. 14, 2022

Related U.S. Application Data
(60) Provisional application No. 63/042,079, filed on Jun. 22, 2020.

(51) Int. Cl. G16H 40/63; A61N 1/372

(52) U.S. Cl. CPC G16H 40/63; A61N 1/372

(57) ABSTRACT

Examples disclosed herein are relevant to selectively bypassing a verification stage in controlling a device based on a received command. Verification can be bypassed when certain conditions are met, such as the occurrence of one or more scenarios. Disclosed techniques can be applied to any of a variety of devices, such as those that use a verification stage in addition to a command stage.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In a first example, there is a method that includes: monitoring sensor data for a pre-defined command; determining whether to require a verification process; and based on the determining, controlling a medical device based on the pre-defined command.

In a second example, there is a system that includes one or more processors configured to: obtain input defining a command; control a medical device based on the command; selectively perform a verification process prior to controlling the medical device based on the input; and bypass the verification process responsive to detecting an occurrence of one or more scenarios.

In a third example, there is an apparatus comprising: a stimulator; a sensor; and one or more processors. The one or more processors can be configured to: stimulate a system of a recipient using the stimulator; receive an input from the sensor; determine whether the input passes a verification process based on the input including a wake input; detect occurrence of at least one bypass scenario; and control the stimulation based on the input responsive to either: the input including a command proximate the wake input; or the at least one bypass scenario occurring.

In a fourth example, there is a computer-readable medium having instructions stored thereon that, when executed by one or more processors, cause the one or more processors to: obtain an input comprising a command; determine whether a bypass scenario occurred; responsive to failing to determine that the bypass scenario occurred, require verification prior to executing the command; and control a medical device based the command.

### BRIEF DESCRIPTION OF THE DRAWINGS

The same number represents the same element or same type of element in all drawings.

FIG. 1 illustrates a system and associated method for selectively bypassing a verification stage in controlling a medical device.

FIG. 2 illustrates a first example method for selectively bypassing a verification stage in controlling a medical device.

FIG. 3 illustrates a second example method for selectively bypassing a verification stage in controlling a medical device.

FIG. 4 illustrates a third example method for selectively bypassing a verification stage in controlling a medical device.

FIG. 5 illustrates an operation that includes obtaining input defining a command and which can also include various additional operations.

FIG. 6 illustrates an operation that includes determining whether to require a verification process and which can also include various additional operations.

FIG. 7 illustrates an operation that includes performing a verification process and which can also include various additional operations.

FIG. 8 illustrates an operation that includes controlling a medical device based on a command and which can also include various additional operations.

FIG. 9 illustrates example scenarios.

FIG. 10 illustrates a functional block diagram of an implantable stimulator system that can benefit from the technologies described herein.

FIG. 11 illustrates a cochlear implant system that can benefit from use of the technologies disclosed herein.

FIG. 12 illustrates a percutaneous bone conduction device that can benefit from use of the technologies disclosed herein.

FIG. 13 illustrates a retinal prosthesis system that comprises an external device, a retinal prosthesis, and a mobile computing device.

### DETAILED DESCRIPTION

Medical devices can receive commands provided by a user, such as a recipient of the medical device or a caregiver. In some examples, the commands are received via the medical device itself (e.g., via a button or touchscreen thereof) or an additional device, such as a remote control, a magnet, or a consumer electronics device (e.g., a phone, tablet, or smart watch). In further examples, medical devices (e.g., implanted medical devices) receive commands through sounds or physical events created by the recipient or a caregiver, such as by knocking on the recipient's head, whistling, teeth clicking, tongue clicking, tapping a microphone, other techniques, or combinations thereof. A specific example of commanding a medical device through knocking a finger knuckle joint on one's own skull is described in U.S. Pat. No. 8,634,918, which is titled “Medical Implant with Safety Features”, issued Jan. 21, 2014, and is hereby incorporated by reference in its entirety for any and all purposes. In another specific example, an audio signal processing unit recognizes control commands from user-generated non-speech sound events in WO 2011/095229, which is titled “Fully Implantable Hearing Aid”, was filed Feb. 8, 2010, and is hereby incorporated by reference in its entirety for any and all purposes.

A verification stage can be implemented to reduce the occurrence of a detected command being a false positive. For instance, passing verification is required prior to the performance of the command that the device detected.

Technology disclosed herein includes technology for selectively bypassing a verification stage in controlling a device. For example, verification is bypassed when certain conditions are met. In scenarios where a false positive is unlikely, a verification stage may not be needed. Further, the risk of a false positive may be preferred over completion of a verification stage in certain circumstances. Scenarios in which a verification step may be unnecessary can include a low-false-positive scenario, a consistent-context scenario, a consistent-behavior scenario, an activity scenario, other scenarios, or combinations thereof.

In an example implementation, a processor monitors sensor data (e.g., data from one or more microphones or accelerometers) for pre-defined sequences, such as a command sequence or wake input. Then, if a wake input is typically required but a command is detected, the command is executed if one or more of the scenarios are also detected. Otherwise, the command is disregarded. In addition or instead, if a command is detected and one or more of the scenarios are also detected, the command is executed without a confirmation sequence that would otherwise be required. In addition or instead, if one or more of the conditions described above are detected, a component is altered to detect command sequences rather than wake inputs. In addition or instead, if one or more of the conditions are not detected, a component is altered to detect command sequences and not wake inputs.

Disclosed techniques can be applied to any of a variety of devices, such as those that require verification before performing a received command. Example devices include medical devices, such as sensory prostheses (e.g., auditory prostheses and visual prostheses), drug pumps, hearing aids, or consumer electronic devices coupled to a medical device. Disclosed examples are applicable to other devices as well, such as personal sound amplification products. Any of a variety of different predetermined commands can be detected, such as those that control basic operations of the medical device. Commands can include commands to request assistance, respond to a phone call, or control a separate device, among others.

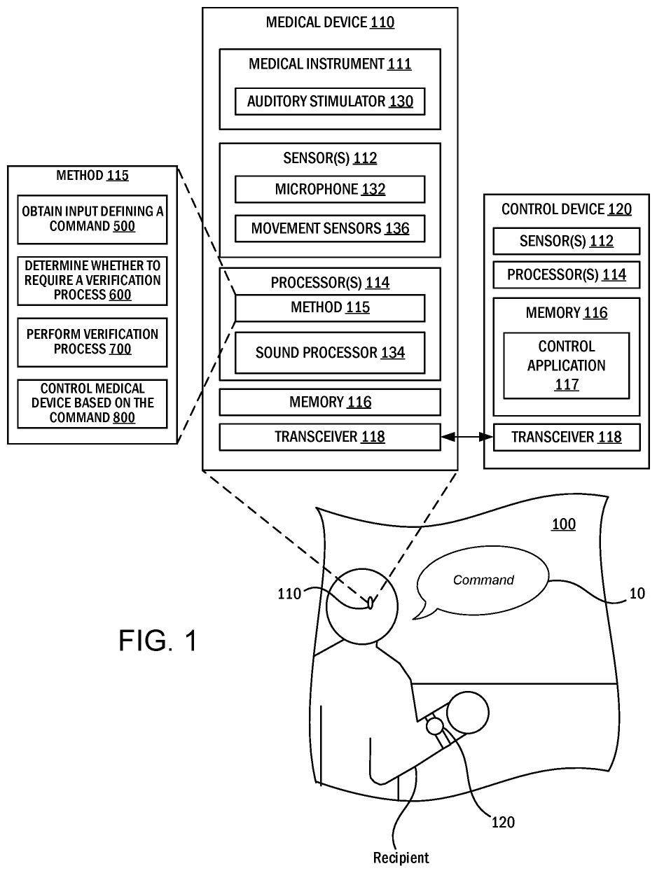

An example implementation of a system and method implementing the selective bypass of a verification stage in controlling a medical device with a command is shown in FIG. 1.

### Example System and Method

FIG. 1 illustrates a system 100 and associated method 115 for selectively bypassing a verification stage in controlling a medical device 110 with a command 10.

A command 10 can be an action that the medical device 110 interprets as an instruction to perform a specific action. The command 10 can be one of a variety of different predefined actions that the medical device 110 is configured to detect and to which the medical device 110 is configured to respond.

The medical device 110 can be a device configured for a medical purpose, such as the diagnosis, treatment, or prevention of a medical condition of a recipient of the medical device 110. The medical device 110 is, for example, a sensory prosthesis (e.g., a visual prosthesis or an auditory prosthesis), a drug pump, a neuromodulation device, a stimulator (e.g., stimulation for tinnitus management), a sleep apnea management device, seizure therapy device, a seizure identification device, or a vestibular implant (e.g., providing vestibular stimulation for balance management), among other devices. The medical device 110 can include any of a variety of components depending on its configuration. As illustrated, the medical device can include a medical instrument 111, one or more sensors 112, one or more processors 114, memory 116, and a transceiver 118.

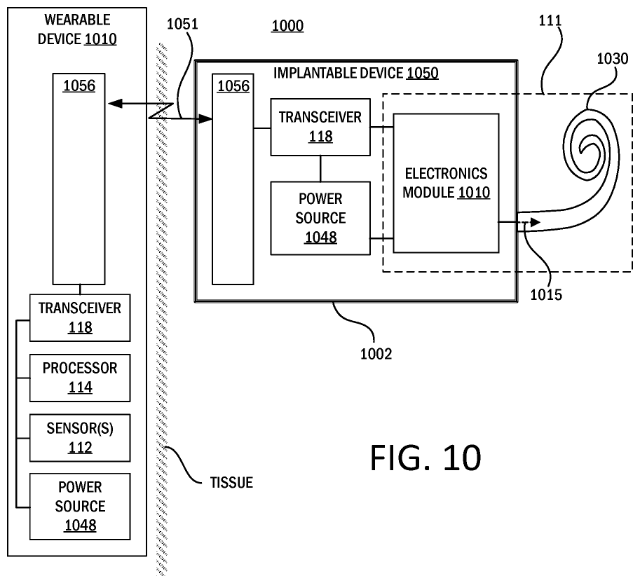

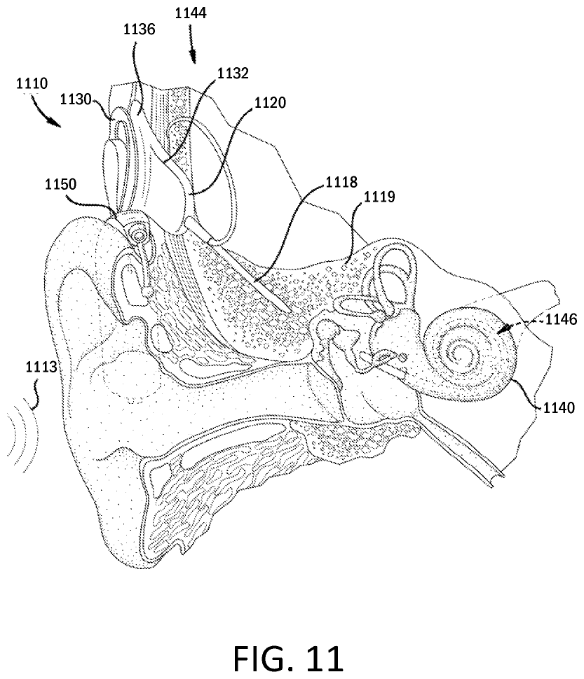

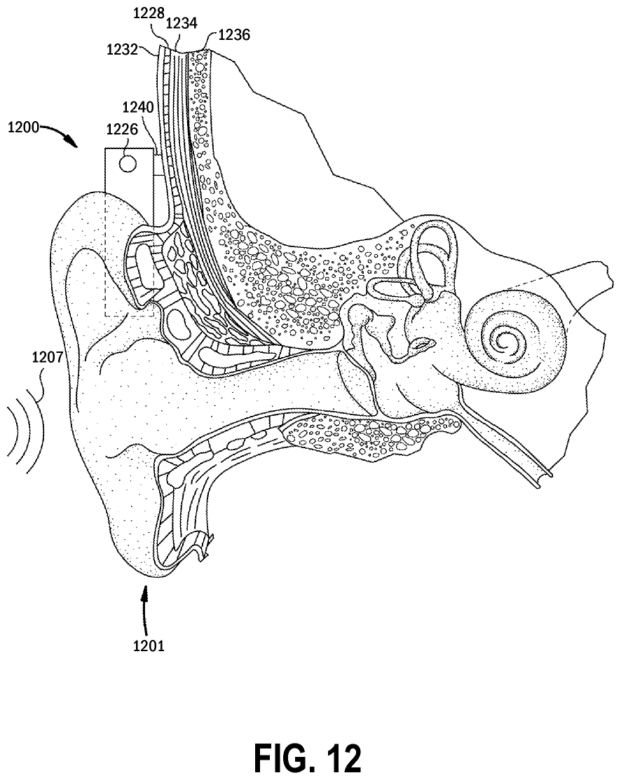

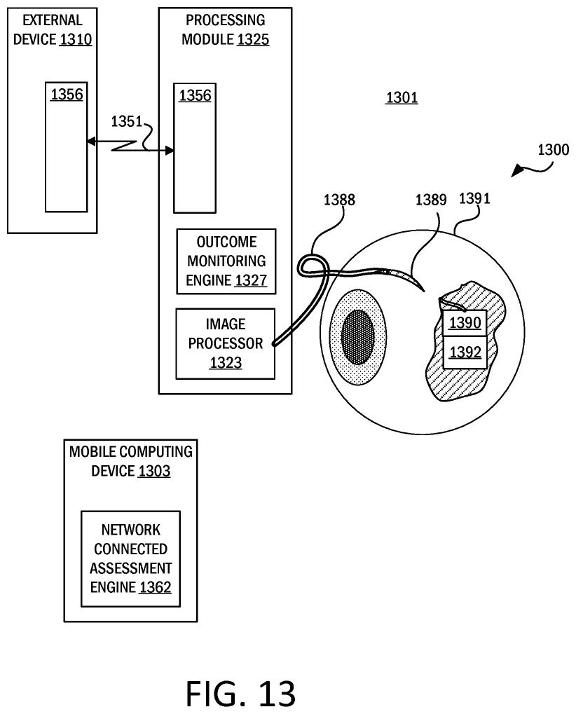

The medical instrument 111 can be one or more components of the medical device 110 configured to perform one or more medical functions of the medical device 110. For example, where the medical device 110 is a stimulator, the medical instrument 111 includes stimulation generation and delivery components as well as additional components. Examples include the electronics module 1010 and stimulator assembly 1030 described in FIG. 10, the stimulator unit 1120 and elongate lead 1118 described in FIG. 11, the actuator of FIG. 12, and the sensor-stimulator 1390 of FIG. 13, which are each described in more detail below. As a specific example, the medical instrument 111 is or includes an auditory stimulator 130.

The auditory stimulator 130 can be a component configured to provide stimulation to a recipient's auditory system to cause a hearing percept to be experienced by the recipient. Examples of components usable for auditory stimulation include components for generating air-conducted vibrations, components for generating bone-conducted vibration, components for generating electrical stimulation, other components, or combinations thereof. Examples can include the electronics module 1010 and stimulator assembly 1030 described in FIG. 10, the stimulator unit 1120 and elongate lead 1118 described in FIG. 11, and the actuator of FIG. 12, which are each described in more detail below.

The sensors 112 are one or more components that generate signals based on sensed occurrences, such as data regarding the environment around the sensors 112, which can include data regarding the recipient, the medical device itself, or the environment around the recipient. The sensors 112 can include one or more components, such as one or more location sensors, telecoils, cameras, pupilometers, biosensors (e.g., heart rate or blood pressure sensors), otoacoustic emission sensors (e.g., configured to provide otoacoustic emission signals), EEG (electroencephalography) sensors (e.g., configured to provide EEG signals), one or more lights sensors (e.g., configured to provide signals relating to light levels), other components or combinations thereof. The sensors 112 can include components disposed within a housing of a containing device as well as components separate from and electrically coupled to the medical device 110 (e.g., via wired or wireless connections).

In examples, the sensors 112 include one or more remote devices connected to the medical device 110 via an FM (Frequency Modulation) connection, such as a remote microphone, a television audio streaming device, or a phone clip device, among other devices having FM transmission capabilities. The sensors 112 can further include sensors that obtain data regarding usage of the medical device 110, such as software or hardware sensors operating on the medical device 110 that track data such as: when the medical device 110 is worn by the recipient, when the medical device 110 (e.g., an external portion thereof) is removed from the recipient, and a current mode in which the medical device 110 is operating, among other data.

In examples, the sensors 112 can include a scene classifier. In some examples, one or more of the processors 114 are configured to act as the scene classifier, which can also act as one or more of the sensors 112. A scene classifier is software that obtains data regarding the environment proximate the medical device 110 (e.g., from one or more of the sensors 112) and determines a classification of the environment. The classifications can be used to determine settings appropriate for the environment. For example, where the medical device 110 is an auditory prosthesis, the scene classifier obtains data regarding the sonic environment around the auditory prosthesis and classify the sonic environment into one or more of the following possible classifications: speech, noise, and music, among other classifications. The medical device 110 can then use the classification to automatically alter the sensory prosthesis settings to suit the environment. For example, where the medical device 110 is an auditory prosthesis, responsive to the scene classifier determining that the sonic environment around the medical device 110 is windy, a wind-noise scene is selected, which modifies settings of the medical device 110 to lessen wind noise. In another example, the scene classifier determines that music is occurring nearby and automatically modifies settings of the medical device 110 to improve musical reproduction. An example scene classifier is described in US 2017/0359659, filed Jun. 9, 2016, and entitled “Advanced Scene Classification for Prosthesis”, which is incorporated by reference herein in its entirety for any and all purposes. Such scenes can be changed automatically by the medical device 110 itself or by a command provided by the recipient.

The sensors 112 can produce sensor data. Sensor data is data produced by a sensor of the sensors 112. Sensor data can take any of a variety of different forms depending on the configuration of the sensor 112 that produced the sensor data. Further, the form and character of the sensor data can change as the sensor data is used and moved throughout the system 100. For example, sensor data begins as a real-time analog signal that is converted into a real-time digital signal within a sensor 112, which is then transmitted in real-time as packets of data to a processor or memory for batch sending (e.g., non-real-time) to another component or device. Additionally, the sensor data can be processed as the sensor data is used and moved throughout the system 100. For instance, the sensor data is converted into a standardized format and have relevant metadata attached (e.g., timestamps, sensor identifiers, etc.).

In the illustrated example, the sensors 112 include one or more microphones 132. A microphone 132 can be a transducer that converts acoustic energy into electric signals. The microphone 132 can include one or more microphones implanted in the recipient or microphones external to the recipient. The microphones 132 can be configured to receive sounds produced external to the recipient. One or more of the microphones 132 can include or be configured as body noise sensors configured to sense body noises produced by the recipient.

In some examples, the sensors 112 further includes one or more movement sensors 136, which can be transducers that convert motion into electrical signals. The movement sensors 136 include, for example, one or more accelerometers and gyroscopic sensors.

In some examples, the sensors 112 further include one or more electrodes configured to detect electrical signals. In some examples, the electrode sensors are electrodes of a stimulator or sensing assembly of the medical instrument 111. The electrode sensors can include internal or external electrode sensors. In an example, the electrode sensors are wearable electrodes, such as via a headband.

The one or more processors 114 can be electronic circuits that perform operations to control the performance of or be controlled by other components of the medical device 110 or the system 100. For example, the processors 114 include one or more microprocessors (e.g., central processing units) or microcontrollers. In certain examples, the one or more processors 114 are implemented as one or more hardware or software processing units that can obtain and execute instructions. The processors 114 can be configured to perform the method 115. In an example, the processors 114 are connected to the memory 116 having instructions encoded thereon that configure the processors 114 to perform the method 115. For instance, the memory 116 can include instructions that, when executed by the one or more processors 114 cause the one or more processors 114 to perform one or more of the operations described herein in association with the method 115. In some examples, the one or more processors 114 include or act as a sound processor 134.

The sound processor 134 can be a set of one or more components that detect or receive sound signals and generate output signals based thereon for use in stimulating a recipient's auditory system (e.g., via the medical instrument 111). The sound processor 134 can perform sound processing and coding operations to convert input audio signals (e.g., generated by the microphone 132) into output signals (e.g., thereby implementing a sound processing pathway) used to provide stimulation via the auditory stimulator 130.

The memory 116 can be one or more software- or hardware-based computer-readable storage media operable to store information. The memory 116 can be accessible by one or more of the processors 114. The memory 116 can store, among other things, instructions executable by the one or more processors 114 to cause performance of operations described herein. In addition or instead, the memory 116 can store other data. The memory 116 can be volatile memory (e.g., RAM), non-volatile memory (e.g., ROM), or combinations thereof. The memory 116 can include transitory memory or non-transitory memory. The memory 116 can include one or more removable or non-removable storage devices. The memory 116 can include RAM, ROM, EEPROM (Electronically-Erasable Programmable Read-Only Memory), flash memory, optical disc storage, magnetic storage, solid state storage, or any other memory media usable to store information for later access. In examples, the memory 116 encompasses a modulated data signal (e.g., a signal that has one or more of its characteristics set or changed in such a manner as to encode information in the signal), such as a carrier wave or other transport mechanism and includes any information delivery media. The memory 116 can include wired media such as a wired network or direct-wired connection, and wireless media such as acoustic, RF, infrared and other wireless media or combinations thereof.

The transceiver 118 can be a component configured to communicate with another device. For example, the medical device 110 includes the transceiver 118 to wirelessly communicate with the medical device 110. The transceiver 118 can provide wireless network access and can support one or more of a variety of communication technologies and protocols, such as ETHERNET, cellular, BLUETOOTH, inductive, near-field communication, and RF (Radiofrequency), among others. The transceiver 118 can include one or more antennas and associated components configured for wireless communication according to one or more wireless communication technologies and protocols. In some examples, the transceiver 118 is configured for wireless transcutaneous communication between an implanted medical device and an external device. Multiple transceivers 118 can be used. For example, different transceivers 118 are used to communicate over different protocols.

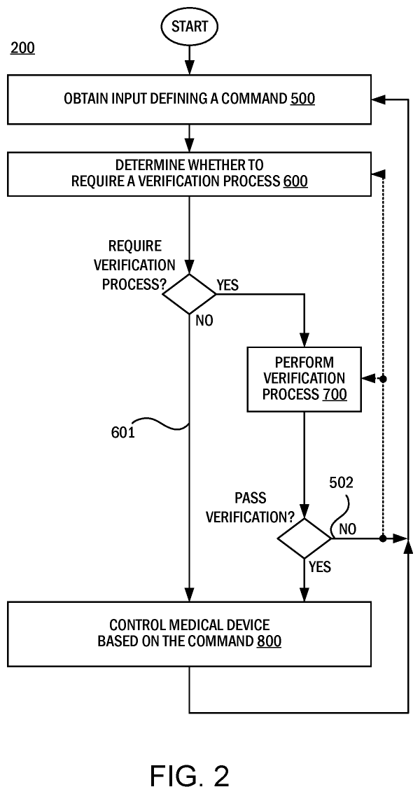

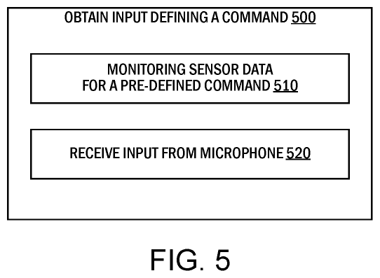

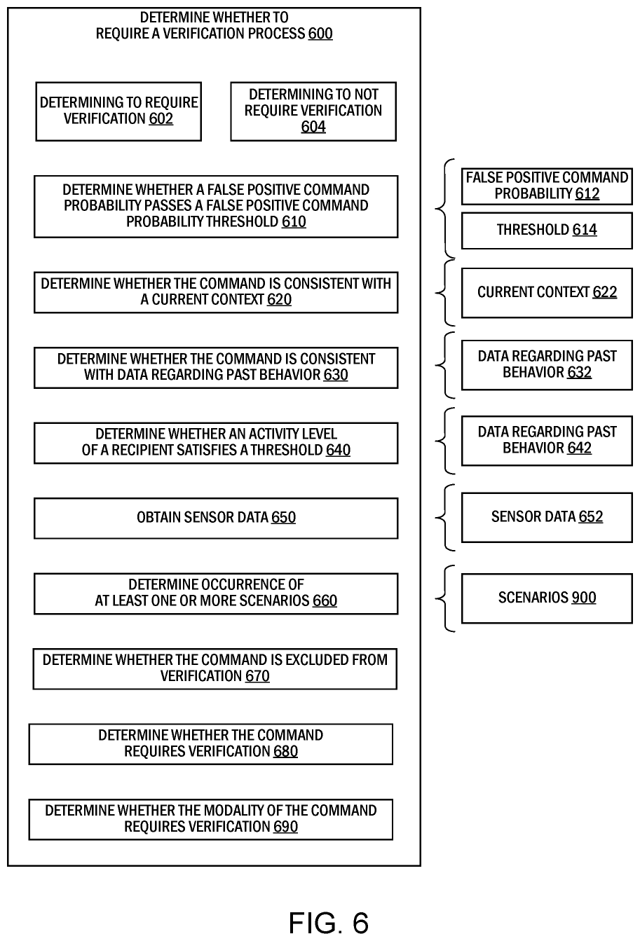

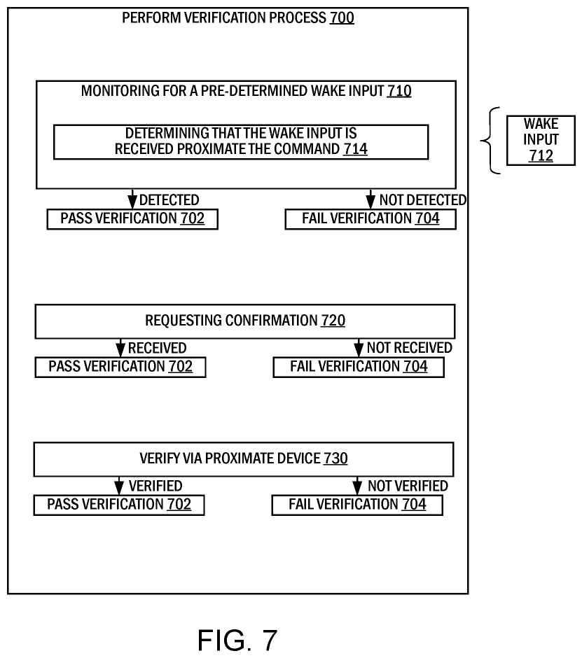

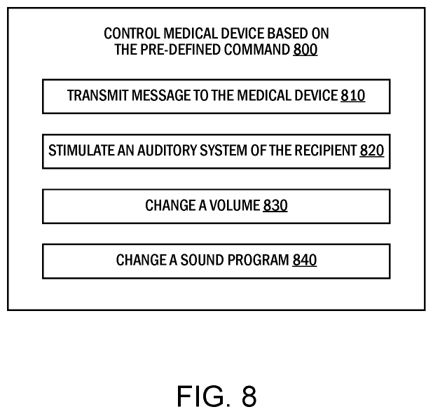

The method 115 can include various operations, including operations 500, 600, 700, and 800. Operation 500 includes obtaining input defining a command 10, which is described in more detail in FIG. 5. Operation 600 includes determining whether to require a verification process, which is described in more detail in FIG. 6. Operation 700 includes performing a verification process, which is described in more detail in FIG. 7. Operation 800 includes controlling a medical device based on the command 10, which is described in more detail in FIG. 8. The operations of the method 115 can be arranged in any of a variety of ways, including methods 200, 300, and 400 and respectively shown in FIGS. 2-4. The method 115 and the operations thereof can be performed by a single device or by multiple different devices acting independently or cooperatively. For instance, the medical device 110 can perform one or more operations of the method 115 in certain implementations. A computing device (e.g., a consumer computing device) can perform one or more operations of the method 115 in certain implementations.

As illustrated, the system 100 also includes a control device 120, which can perform one or more operations of the method 115. The control device 120 is a device that can facilitate the control of the medical device 110. The control device 120 can take any of a variety of different forms. As illustrated, the control device 120 can include one or more sensors 112, processors 114, memory 116, and one or more transceivers 118, such as described above. In some examples, the control device 120 is a consumer electronics device, such as a phone, tablet, smart watch, or heart rate monitor, among other forms. The consumer electronics device can be a computing device owned or primarily used by the recipient of the medical device 110 or a caregiver for the recipient. In some examples, the control device 120 operates as a remote control for the medical device 110, allowing a user to provide commands to the medical device 110 using the control device. In addition or instead, the control device 120 can act as a key to permit the changing of operations of the medical device 110. For example, the medical device 110 is configured to prohibit changing settings of the medical device except for when the control device 120 is used to unlock the functionality, such as by bringing the control device 120 proximate the medical device 110.

In some examples, the control device 120 implements a control application 117. The control application 117 can be a computer program stored as computer-executable instructions in memory 116 of the control device 120 that, when executed, performs one or more tasks relating to the system 100. The control application 117 can cooperate with the medical device 110. For instance, the control application 117 can control when and how function is provided by the medical instrument 111. In some examples, such control is performed automatically by the control application 117 or based on input received from a user of the control device 120.

The components of the system 100 can cooperate to perform the operations of the method 115. In some examples, some or all of the operations are performed by a device connected to the medical device 110 (e.g., the control device 120). Example implementations arrangements of the operations of the method 115 are described in FIGS. 2-4.

### Example Method Arrangements

FIG. 2 illustrates an example method 200, which is an example arrangement of the operations of method 115 for selectively bypassing a verification stage in controlling a medical device 110 with a command 10. Generally, the method 200 arranges the method 115 to determine whether to require a verification process after obtaining the input defining the command 10. This can permit the verification process to take into account the content of the command 10 to determine whether to require verification. In an example, the verification process includes receiving confirmation from the recipient that the command 10 was correctly interpreted by the system 100 (e.g., the recipient said “volume up” but the system 100 incorrectly interpreted the phrase to mean the equivalent of “volume off”). Controlling the medical device 110 based on the command 10 (operation 800) can be performed responsive to receiving the confirmation that the pre-defined command 10 is to be performed. As illustrated, the method 200 can begin with operation 500, which includes obtaining input defining a command 10. Following operation 500, the flow of the process can move to operation 600, which includes determining whether to require a verification process. If a verification process is determined to be required, the flow of the method 200 moves to operation 700. If a verification process is determined to be not required, the flow of the method bypasses (which can be referred to as operation 601) the verification process and proceeds to operation 800. Following operation 700, which includes performing the verification process, the flow of the method 200 can move to operation 800 if the verification process is passed or return to operation 500, otherwise. By failing to pass the verification, the return to operation 500 can result in preventing controlling the medical device 110 based on the command (which can be referred to as operation 502). In some examples, failing the verification process results in the flow of the method 200 returning to operation 600 or 700 for reassessment (e.g., in view of new information). For instance, the circumstances may have changed or the verification was too difficult and the recipient may want the original command to be executed without the recipient needing to provide the command again.

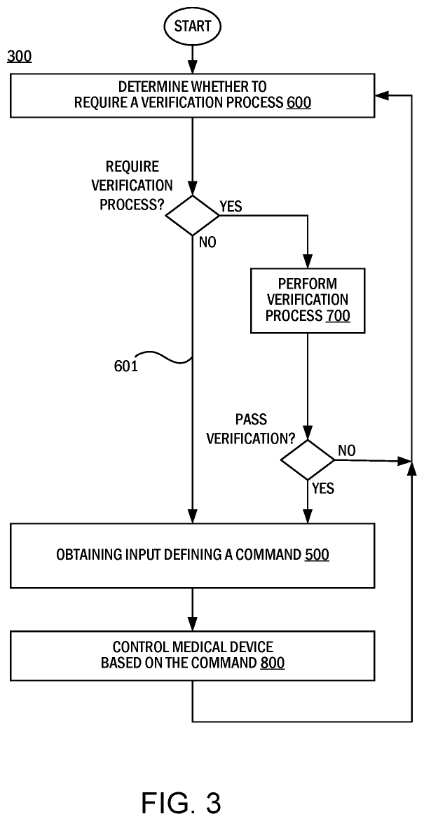

FIG. 3 illustrates an example method 300, which is another example arrangement of the operations of method 115. Generally, the example method 300 is a variant in which the determining whether to require a verification process and the verification process (if performed) occur before obtaining input defining a command 10. For example, such a method 300 includes performing the obtaining input operation 500 responsive to detecting a predetermined wake input in the verification process (see, e.g., operation 710 of FIG. 7, infra). The method 300 can begin with operation 600 in which it is determined whether to require a verification process. If the verification process is determined to be required, then the flow of the method 300 can move to operation 700, which includes performing the verification process. Following operation 700, if the verification process was not passed, then the flow of the process can return to operation 600. If the verification passes or verification is determined to not be required (thereby being operation 601 to bypass the verification process), then the flow of the method 300 can move to operation 500. Operation 500 includes obtaining input defining a command 10. Following operation 500, the flow of the method 300 can move to operation 800, which includes controlling the medical device 110 based on the command. After operation 800, the flow of the method 300 can return to operation 600.

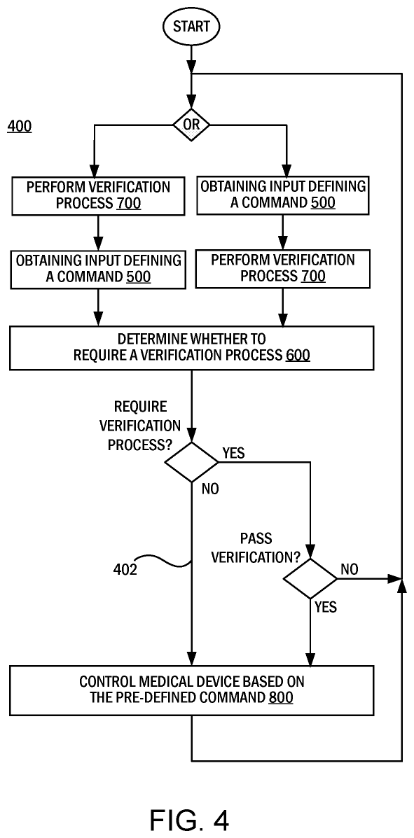

FIG. 4 illustrates an example method 400, which is yet another example arrangement of the operations of method 115. Generally, the example method 400 is a variant in which it is determined whether to require verification after the verification is already performed. For example, the outcome of the verification process is ignored in certain circumstances. The method 400 can begin with performing the verification (operation 700) or with obtaining input defining a command (operation 500), with the other operation being performed next or simultaneously. Following the completion of operations 500 and 700, the flow of the method 400 can move to operation 600, in which it is determined whether to require a verification process. If a verification process is required and not passed, the flow of the method 400 can return to the start. If the verification process is not required, then the method 400 permits (which can be described as operation 402) operation 800 to be performed regardless of the outcome of the verification process. If the verification process is determined to be required and passes, then the flow of the method 400 moves to operation 800. Following operation 800, the flow of the method 400 can return to the start of the method.

The methods 200, 300, 400 of FIGS. 2-4 are provided for example purposes. Other variants are also possible. Further, additional operations can be performed. For instance, at various points in the process, there can be tests for determining whether to timeout the process. For example, the determinations (e.g., that verification passes) are valid for a predetermined amount of time, after which the determination is no longer valid and would need to be performed again. Additional details regarding each of the operations is provided in more detail in the following figures.

### Obtaining Input Defining a Command

FIG. 5 shows operation 500, which includes obtaining input defining a command 10 and which can also include various operations. The obtaining can be from any of a variety of modalities and can be obtained using one or more of the sensors 112. The commands 10 can be provided over one or more of various modalities. The modalities can include audio, visual, tactile, gestural, magnetic, electronic, other modalities, or combinations thereof. An audio command can include a command spoken by a person that can be detected by a microphone and analyzed (e.g., using natural language processing) to determine the meaning (or at least an action to be taken) specified by the command. Other audio commands can include non-verbal sounds that can be determined to be interpreted as commands. For example, the occurrence of particular sounds (e.g., a whistle sound produced by a recipient or a tone sound produced by a control device 120) are understood by the medical device 110 as being a command 10. The commands in the audio modality can, but need not be, within a range of human hearing (e.g., can include infrasonic and ultrasonic signals). Sensors 112 that can detect commands in the audio modality can include microphones 132. A visual command 10 can include a command 10 conveyed visually. For instance, a command 10 can be conveyed through a machine-readable code (e.g., a bar code or a QR code), patterns of light (e.g., particular patterns of flashing light), or patterns of blinks by the recipient. Visual commands can be detected through sensors 112 configured to detect activity in the visual modality (e.g., a camera or light sensor). In some examples, the visual commands are conveyed through frequencies beyond the range of typical human detection (e.g., infrared). Tactile commands can include commands provided by contact or vibrations, such as through taps, swipes, or knocks, among others. Tactile commands can be detected through vibration sensors (e.g., accelerometers or microphones) or contact sensors (e.g., capacitive sensors). Gestural commands can include commands that are provided through movement. For example, a recipient moves their arm, hand, or body, which is detected and the gesture provided thereby can convey a command. Sensors 112 that can detect commands in the gestural modality can include motion sensors (e.g., accelerometers and gyroscopes) and visual sensors (e.g., a camera providing inside-out tracking or outside-in tracking). Magnetic modalities can include the presence or absence of one or more magnets or particular magnetic strengths that correspond to particular commands. Electronic modalities can include modalities that use electronic signals to convey a command. For instance, the electronic signal can be a wirelessly communicated data packet (e.g., using WI-FI, BLUETOOTH, or other protocols) that describes a message. Other modalities can include, microwave communication and radio wave communication.

Certain commands can be detected through any of a variety of modalities and the medical device 110 can be configured to detect the command 10 through one or more modalities. For instance, a recipient knocking on their own head with their knuckles can be detected through audio (e.g., the sound caused by the tapping), tactile (e.g., the vibrations caused by the tapping), or gestural (e.g., the knocking motion itself). A particular pattern of knocks can correspond to a particular command. The recipient whistling can be detected primarily through an audio modality. A recipient clicking their teeth or tongue can be primarily detected through an audio modality. Another device being brought in proximity to the medical device 110 can provide commands through the electronic modality (e.g., using data packets to transmit commands), audio modality (e.g., by playing particular sounds), visual modality (e.g., by displaying a particular pattern on a screen), magnetic modality (e.g., by the device generating a magnetic field), or tactile modality (e.g., by the device generating particular vibrations), among techniques or combinations thereof.

Any of a variety of different commands 10 can be provided, such as those that control basic operations of the medical device 110. Commands 10 can include commands to request assistance, respond to a phone call, or control a separate device, among others. A medical device 110 can distinguish between different commands 10 (or lack of commands) through an analysis of data produced by one or more sensors 112 monitoring the relevant one or more modalities.

Example commands 10 can control functions of the medical device 110, such as administering therapy (e.g., stimulation or drug deliver), changing an intensity of stimulation (e.g., volume), muting the medical device 110, switching between different modes of medical device operation (e.g., switching from an external hearing mode to an invisible hearing mode or vice versa), activating a sleep mode, deactivating a sleep mode, deactivating a data logging mode, causing a play command, causing a pause command, modifying noise reduction (e.g., starting or stopping noise reduction or increasing or decreasing an aggressiveness of noise reduction. Commands 10 can cause the medical device 110 to provide status information regarding the medical device 110, such as a current battery level of the medical device 110, a currently-running program of the medical device 110, or a next scheduled appointment for the recipient of the medical device 110.

The operation 500 can include operation 510 and operation 520, among other operations.

Operation 510 includes monitoring sensor data for a pre-defined command. For example, the pre-defined command 10 is from a recipient of the medical device 110. In an example, monitoring the sensor data for the pre-defined command 10 from the recipient of the medical device 110 includes monitoring for a voice command 10 or a tap command. The sensor data can be obtained from one or more of the sensors 112. The sensor data can be monitored for the predetermined commands 10 in any of a variety of ways depending on the modality and the predefined commands. In some examples, the command 10 is determined to exist based on values in the sensor data passing a threshold or changing, such as may be the case where the command 10 is provided over a button or switch. In examples, the command 10 is determined to exist based on a particular pattern in the data. In examples, the command 10 is determined to exist based on the output of one or more analysis algorithms or techniques, such as natural language processing.

Operation 520 includes receiving an input from the microphone 132. For example, the input received from the microphone 132 is analyzed to determine whether the input has a particular pattern. In some examples, the input is processed by applying a speech-to-text technique and then analyzing the resulting text using natural language processing to determine whether the input includes a command. In some examples, the input from the microphone is analyzed for particular frequencies or patterns that correspond to predetermined commands 10.

### Determining Whether to Require a Verification Process

FIG. 6 shows operation 600, which includes determining whether to require a verification process and which can also include various operations. The operation 600 can include and be based on operations 602, 604, 610, 620, 630, 640, 650, 660, 670, 680, and 690, among other operations.

Operation 602 includes determining to require the verification process, and operation 604 includes determining to not require the verification process. The determination to require or not require the verification process can be based on the performance of one or more other operations. The determination can be the result of an output of such operations. The determination can be represented by an output of a relevant component. For example, the determination is represented by an output of a software function that indicates that the verification process is or is not required. In addition or instead, the determination is represented by an output of an electrical circuit.

Operation 610 includes determining whether a false positive command probability 612 passes a false positive command probability threshold 614. This operation can include determining the false positive command probability 612 and then comparing the determined probability 612 to the false positive command probability threshold 614. For example, the false positive command probability threshold 614 is a predetermined (and optionally configurable) threshold value against which the false positive command probability 612 is compared. In certain implementations, the false positive command probability threshold 614 is a value such that when the false positive command probability 612 indicates that the command 10 is more likely than not a false positive, the false positive command probability threshold 614 is passed. The false positive command probability 612 can be determined in any of a variety of different ways. For example, the probability 612 can be determined based on how far the command 10 deviates from a pure signal that would cause the command 10 to be triggered. For example, the device 110 can be configured to determine that detecting an audio tone of 5 Hz for three seconds corresponds to a power off command. The input received from a sensor 112 can indicate that a tone of 5 Hz was received for 2.5 seconds. In some embodiments, the difference between the detected signal and the signal that triggers the command 10 is 0.5 seconds and the false positive command 10 probability 612 is determined based on this difference. In some examples, the operation is based on the value itself or a value generated based on the value (e.g., a percent difference between the actual value received and the value associated with triggering the command. In some examples, the false positive command probability 612 is based on a modality over which the command 10 is received. For instance, certain kinds of input can be labeled as more reliable and thereby have a lower false positive command probability 612, such as physical button input or input received from another device (e.g., a control device 120). In an example, responsive to determining that the recipient is sleeping, the detected command can be determined to be a false positive. In another example, where the device 110 is configured for use while the recipient is sleeping (e.g., a sleep apnea device), commands received while the recipient is awake can be determined to be likely to be true positives. In examples, determining that the false positive command probability 612 passes a false positive command probability threshold 614 is an indication that verification should be required. Further, in such examples, determining that false positive command probability 612 does not pass the false positive command probability threshold 614 is an indication that verification should not be required.

Operation 620 includes determining whether the command 10 is consistent with the current context 622. The current context 622 can take any of a variety of forms. For example, the context 622 can be a context within the recipient (e.g., based on a recipient's heart rate) or the environment around the recipient (e.g., high ambient noise). In an example, determining whether the command 10 is consistent with the current context 622 includes determining whether the command 10 is expected or unexpected given the current context 622. In some examples, determining that the command 10 is consistent with the current context 622 can be based on the command 10 being to decrease a volume level of the medical device 110 and that the input was received in a noisy environment. In some examples, determining that the command 10 is consistent with the current context 622 is based on the command 10 being to increase a volume level of the medical device 110 and that the input was received in a quiet environment. In some examples, determining that the command 10 is consistent with the current context 622 is based on the command 10 being to change an operating mode of the medical device 110 and a sound environment of the medical device 110 changed within a threshold amount of time. In some examples, determining that the command 10 is consistent with the current context 622 can be based on the command 10 being to deactivate the medical device 110 and an output of the medical device 110 being higher than an output threshold. The system 100 can store data regarding commands 10 that are or are not expected to be received during a particular context. Commands that are expected to be received during the particular context can be determined to be consistent with the context. Commands that are not expected to be received during the particular context can be determined to be inconsistent with the context. The detected command 10 can then be compared with the defined commands that are or are not expected to be received from that context. In some examples, responsive to the command 10 being determined to be inconsistent with the current context 10, verification is required (operation 602) and responsive to the command 10 being determined to be consistent with the current context 622, verification can be not required (operation 604). In some examples, the current context 622 includes temporal information. For example, the command 10 is a command to deliver therapy and the context can be based on a schedule for delivering therapy.

Operation 630 includes determining whether the command 10 is consistent with data regarding past behavior 632. The past behavior can be a behavior of the recipient, a caregiver, or a medical professional. In an example, the operation 630 includes determining that the command 10 is consistent with commands previously provided by a recipient when at a certain physical location, connected to a certain device, or in a certain audio environment as determined based on one or more sensors 112. In some examples, the data regarding past behavior 632 of the recipient (e.g., the commands 10 provided by the recipient or another person) is stored in association with other data (e.g., readings from sensors 112 or determined contexts). This data can be stored, for example, in the memory 116 of the device 110. As a result, a profile can be built that indicates when (e.g., in which contexts) certain commands tend to be received. Received commands 10 can then be compared against the stored data regarding past behavior 632 to determine whether the command 10 is consistent with the past behavior of the recipient. In some examples, responsive to the command 10 being determined to be inconsistent with the data regarding past behavior 632, verification is required (operation 602) and responsive to the command 10 being determined to be consistent with the data regarding past behavior 632, verification need not be required (operation 604).

Operation 640 includes determining whether an activity level 642 of the recipient passes a threshold. The activity level 642 can be based on objective measures of the recipient (e.g., heart rate) or GPS and/or accelerometers (e.g., which may indicate that the recipient is running) in addition to or instead of other activity level determinations. The activity level 642 of the recipient can be determined based on data from the sensors 112. In some examples, the activity level 642 is a physical activity level of the recipient (e.g., the recipient is exercising). In other examples, the activity level is a mental activity level of the recipient (e.g., the recipient is working, studying, or concentrating on driving). In some examples if the recipient's activity level is sufficiently high (e.g., based on a comparison with the threshold), a verification process is not required (operation 604). Alternatively, if the recipient's activity level is sufficiently high, the verification process is required (operation 602). In some examples, whether and how activity level relates to verification is configurable. Sometimes, a high activity level results in an increased risk of false positives due to the nature of the activity level. For instance, noise and motion that accompany the high activity level can cause false positives. Thus, a sufficiently high activity level 642 can warrant requiring a verification process. But during such activities, the recipient may nonetheless accept the risk of false positives and forgo verification to ensure that commands 10 are detected and executed. For instance, the effects of the high activity level may undesirably mask or obscure verification provided by the recipient. Further, while the recipient is engaged in the activity, the recipient may not want their attention diverted (e.g., while biking or driving) or may be unable to readily perform verification (e.g., while showering or exercising). In some examples, a sufficiently low activity level 642 results in verification being required (e.g., due to it being relatively easy for the recipient to provide verification) or verification not being required (e.g., due to the risk of false positives being low due to the low activity level).

Operation 650 includes obtaining sensor data 650. As is clear from the operations described in relation to operation 600, the determination of whether to require the verification process can be based on sensor data obtained from one or more of the sensors 112. The sensor data used for determining whether to require a verification process can be the same as or different from the sensor data monitored for the command. For example, the monitoring sensor data for a pre-defined command 10 in operation 510 includes obtaining first sensor data from a first sensor, and the determining whether to require a verification process in operation 600 can be based on second sensor data obtained from a second sensor.

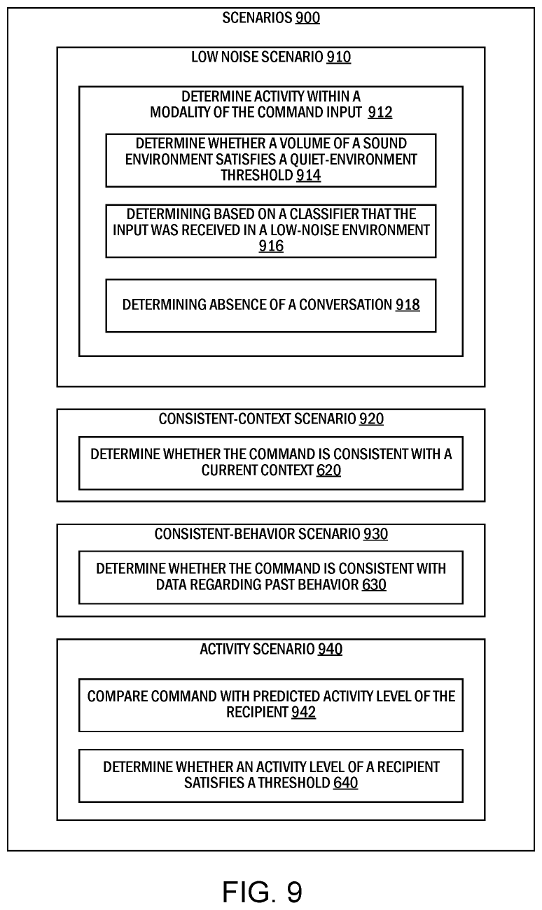

Operation 660 includes determining the occurrence of at least one or more scenarios 900. The scenarios 900 can include bypass scenarios whereby the occurrence of the bypass scenario results in the bypassing a verification process (operation 604) and that failing to detect the occurrence of the scenarios can result in requiring verification (operation 602). In some examples, the operation 600 requires the occurrence of two or more scenarios to determine to not require verification (operation 604). In an example, operation 600 determines to not require a verification process responsive to operation 660 determining that at least two of the scenarios 900 occurred. For instance, requiring the occurrence of multiple scenarios increases the burden required to bypass verification to help ensure that verification truly warrants being bypassed. The determining can be based on the scenarios occurring contemporaneous with the input that included the command 10. Examples of the scenarios 900 are described in more detail in relation to FIG. 9, infra.

Operation 670 includes determining whether the command 10 is excluded from verification. Some commands 10 can be determined to be excluded from verification. The determining can include comparing the command 10 with a set of one or more excluded commands 10 (e.g., a data structure storing such commands can be stored in the memory 116) and if the command 10 is excluded, then the command 10 can be determined to not require verification (operation 604), otherwise the command 10 can be determined to require verification (operation 602). For example, commands 10 that request assistance from a caregiver, medical professional, or emergency services are selected to be excluded from requiring verification. The commands 10 to be excluded from verification can be mutable, such as requests for assistance can be excluded from verification based on a health status of the recipient (e.g., as set manually or determined automatically, such as based on temperature sensors). In an example implementation, requests for assistances require verification under typical circumstances, but when the recipient is determined to be in poor health or determined to have suffered an actual or potential injury (e.g., based on detecting the recipient may have fallen or been in an accident based on data from the sensors 112), the requirement for verification can be bypassed. Where the medical device 110 is a totally-implanted auditory prosthesis, a command 10 excluded from verification can be responding to a phone call. In some examples, the commands 10 are excluded based on accompanying conditions of the recipient or the environment. For example, commands 10 to respond to a phone call via an implant are, in some instances, implemented without verification despite the detected presence of a nearby mobile phone if the implant or another device detects that the recipient is showering or otherwise unable to readily access the phone. As yet another example, commands 10 to control a separate device paired to an implant are, in some instances, implemented without a verification stage despite the possibility that others could be affected by such commands 10.

Operation 680 includes determining whether the command 10 requires verification. For example, certain commands are flagged as always requiring verification. For example, certain commands that can have severe effects on the recipient (e.g., delivering large doses via a drug pump), the device (e.g., placing the device in a potentially damaging state), or others (e.g., contacting emergency personnel) are determined to have such severe consequences that verification is always required before such commands are to be performed. Whether a command 10 requires verification or can be excluded from verification can be based on consequences of a false positive or a false negative of the command. In addition or instead, such commands 10 can be determined to require higher levels of scrutiny prior to deciding to forgo verification. For example, where verification is determined to be bypassed based on the satisfying a first threshold, satisfying a second, a higher threshold is required for certain commands having severe consequences. As another example, the occurrence of additional scenarios is required before determining to bypass verification. In a further example, verification is required to perform an action (e.g., an action having severe effects) absent objective measures of support (e.g., express indication by the recipient or based on sensor data) for the performance of the action.

Operation 690 includes determining whether the modality of the command 10 requires verification. For example some modalities are at relatively higher or lower risk of false positives. Button input provided through physical or virtual buttons of the medical device 110 or the control device 120 can be determined to be of a modality that is sufficiently unlikely to produce false positives as to not require verification.

In some examples multiple different combinations of operations 610, 620, 630, 640, 650, 660, 670, 680, and 690 are used to determine whether to require or not require verification. In some examples, one or more of the operations are required to weigh in favor of not requiring verification and one or more of the operations must not weigh in favor of requiring verification. For instance, requiring multiple different combinations of the operations to be satisfied makes requiring or not requiring verification relatively easier or harder. The relative ease or difficulty in bypassing verification can be modified by a user (e.g., the recipient, a clinician, or a caregiver using a user interface of the control application 117) to tune the verification requirement to match particular preferences. Certain recipients may prefer relatively looser or stricter requirements for requiring verification.

### Performing Verification Process

FIG. 7 shows operation 700, which includes performing a verification process and which can also include various operations. The operation 700 can include operations 702, 704, 710, 720, and 730, among other operations. The operations can be based on data obtained from the sensors 112. The sensor data can be the same as, different from, or in addition to the sensor data used to obtain the input that can include the command 10. In an example, the input is obtained from a first sensor 112 and the verification process can be based on data from a second sensor 112. A verification process can include a passcode-like process, whereby no control of the medical device 110 is possible without the verification process or a simple verification such that the recipient could control the medical device 110 in other ways even if the particular pre-command 10 will not be implemented.

Operation 702 includes passing the verification process, and operation 704 includes failing the verification process. The determination to pass or fail the verification process can be based on the performance of one or more other operations. The determination can be the result of an output of such operations. The determination can be represented by an output of a relevant component. For example, the determination is represented by an output of a software function that indicates that the verification process is or is not passed. In addition or instead, the determination is represented by an output of an electrical circuit.

Operation 710 includes monitoring for a predetermined wake input 712. The wake input 712 can be a predetermined input to the medical device 110 that acts as a wake signal. In an example, the medical device 110 operates in an inactive state with respect to some or all of the functionality of the medical device 110. The inactive state can be a low-power state to conserve resources of the medical device 110. For instance, the medical device 110 operates in an inactive state with respect to receiving commands 10 except for a wake input 712. The use of these different states can conserve resources of the medical device 110 (e.g., by no longer monitoring for various kinds of input), while permitting the medical device 110 to be awakened to receive such commands 10 after receiving the wake input 712. The requirement of a wake input 712 can further and reduce the risk of false positive commands 10. The wake input 712 can be an extra input, distinct from a command 10, that can be used to reduce a chance of a false positive. For instance, the device 110 can rely on a command 10 being provided proximate the wake input 712 as being a true positive command. The wake input 712 can be used as a verification that a command 10 received proximate the wake input 712 is an intended command. For example, the wake input is a particular wake phrase spoken by a user, such as “my implant . . . ” that can serve to activate the medical device 110 to prepare the device 110 for receiving an input indicative of a command 10 (e.g., “ . . . volume up”). The wake input 712 can be received in other modalities than the modality over which the command 10 is provided. For example, the command 10 is provided over an audio input and a particular magnetic field or tactile input can be used as the wake input 712. The receiving of the wake input 712 over a different modality can result in a lower risk of a false positive stimulus being interpreted both as a wake input 712 and as a command 10. For instance, where a single modality (e.g., tap inputs detected as vibrations) is used for both wake inputs 712 and commands 10, sufficiently high noise over the modality (e.g., as a result of the recipient chewing hard food, the vibrations of which are picked up via a vibration sensor 112) might unintentionally be detected as the wake input 712 and further as a command 10. Whereas if different modalities are used for the wake input 712 (e.g., a tap input) and the commands 10 (e.g., voice commands), then the risk of an event causing a false positive wake input also resulting an false positive command is lower.

In an example, operation 710 includes operation 714, which can include determining that the wake input 712 is received proximate the command 10. For example, the wake input 712 is received before the command 10 or after the command 10 but nonetheless proximate the command 10 responsive to the command 10 being received within a threshold amount of time of the wake input 712. For instance, where the wake input is the spoken phrase “my implant” and the command 10 is “sleep”, the phrase “my implant, sleep” has the wake input 712 prior to the command 10. The phrase “sleep, my implant” has the wake input 712 after the command 10. The medical device 110 can be configured to recognize one or both of such situations.

Responsive to the wake input 712 not being detected or the wake input 712 not being detected within a threshold amount of time, the verification can be failed (operation 704). Responsive to the wake input 712 being detected (e.g., detected within a threshold amount of time), the verification can be passed (operation 702).

Operation 720 includes requesting confirmation that the command 10 is to be performed. Responsive to receiving the confirmation, the verification can be passed (operation 702), otherwise the verification can be failed (operation 704). The verification can be via a same or different modality as the modality in which the command 10 was originally received. In an example, the command 10 is to turn off the medical device 110, and the confirmation can be the medical device 110 or the control device 120 asking the user (e.g., visually, audibly, or tactilely) “are you sure you want to turn off the device?” If it is detected that the user responded affirmatively, then the verification can pass (operation 702), otherwise, the verification can fail (operation 704).

Operation 730 includes verifying via a device proximate to the medical device 110. The proximate device can be, for example, the control device 120. In some examples, the proximity is determined based on the presence or absence of a signal (e.g., a magnetic field, an audible signal, or a visual signal) provided by the control device. In some examples, the proximity is determined based on a wireless communication. For instance, the device 110 pings for the other device and, if a response is received, determine that the device is proximate the medical device. If the device is determined to be proximate the medical device 110, then the verification can pass (operation 702), otherwise, the verification can fail (operation 704).

### Control Medical Device Based on the Command

FIG. 8 shows operation 800, which includes controlling a medical device 110 based on the command 10 and which can also include various operations. As described above, the control of the medical device 110 can be based on whether the verification process is required or whether the verification process is bypassed. The performance of operation 800 can be based on executing the command 10 specified by the input. The operation 800 can include operations 810, 820, 830, and 840 among other operations.

Operation 810 includes transmitting a message to the medical device 110. For example, one or more of the operations described herein need not be performed by the medical device 110 itself. At least some of the operations can be performed by another device (e.g., the control device 120). In some examples, some of the operations are performed by another implant. For instance, the medical device 110 is too deeply implanted or otherwise unable to be directly interacted with by the recipient or a control device, so the medical device 110 can be controlled by a separate implant or device.

Operation 820 includes, where the medical device 110 includes the auditory stimulator 130, stimulating an auditory system of a recipient using the auditory stimulator 130. Controlling the medical device 110 can include stimulating the auditory system of the recipient based on the command 10. For example, the command 10 is to begin stimulation, stop stimulation, pause stimulation, increase stimulation intensity, decrease stimulation intensity, or change a mode of stimulation, among other commands or combinations of commands. Examples of auditory stimulation and ways that the commands can affect stimulation can be understood through other disclosures herein, including those of FIGS. 10-12.

Operation 830 includes changing a volume of the medical device 110. In some implementations, the medical device 110 can include a volume parameter that can be changed based on a provided command 10. For example, where the medical device 110 is an auditory prosthesis, a volume corresponding to the intensity of the provided signal is modified by being increased or decreased.

Operation 840 includes changing settings of the medical device 110. The medical device 110 can have any of a variety of different kinds of settings that affect its operation. These settings can be changed by the command 10. Where the medical device 110 is an auditory prosthesis, the medical device 110 can operate according to one or more different auditory prosthesis settings. The auditory prosthesis settings are one or more parameters having values that affect how the medical device 110 operates. For instance, the auditory prosthesis settings can include a map having minimum and maximum stimulation levels for frequency bands of stimulation channels. The mapping is then used by the medical device 110 to control an amount or manner of stimulation to be provided. For instance, where the medical device 110 is a cochlear implant, the mapping can affect which electrodes of the cochlear implant to stimulate and in what amount based on a received sound input. In some examples, the auditory prosthesis settings include two or more predefined groupings of settings selectable by the recipient via providing the command 10. The auditory prosthesis settings can also include sound processing settings that modify sound input before the sound input is converted into a stimulation signal. Such settings can include, for example, particular audio equalizer settings that boost or cut the intensity of sound at various frequencies. In examples, the auditory prosthesis settings include a minimum threshold for which received sound input causes stimulation, a maximum threshold for preventing stimulation above a level which would cause discomfort, gain parameters, loudness parameters, and compression parameters. The auditory prosthesis settings can include settings that affect a dynamic range of stimulation produced by the medical device 110. The modification of settings can affect the physical operation of the medical device 110, such as how the medical device 110 provides therapy to the recipient.

### Scenarios

As described in relation to operation 660 of FIG. 6, the determining whether to require a verification process can be based on whether or not the occurrence of at least one scenario 900 is detected. FIG. 9 shows example scenarios 900 that can be detected. The scenarios 900 can include a low-noise scenario 910, a consistent-context scenario 920, a consistent-behavior scenario 930, and an activity scenario 940, other scenarios, or combinations thereof. The scenarios 900 can be scenarios 900 in which false positive command 10 is unlikely.

The low-noise scenario 910 can be a scenario 900 in which there is a relatively low amount of noise in a sensor modality. Detecting the occurrence of the low-noise scenario 910 can include performing operation 912. Operation 912 can include determining activity within a modality of the input via which commands 10 are received. Where the command 10 is provided by a tactile input (e.g., tap, touch, swipe inputs), a low noise scenario can occur when a recipient is relatively still as can be determined by an accelerometer. For instance, it can be determined that the recipient's head or body is relatively motionless (e.g., the recipient is not engaged in physical activity) while commands 10 are provided, thus there would be relatively low noise in the signal provided by the sensors 112 that detect tactile input. The relatively low noise can result in a relatively lower risk of false positives. Likewise, a relatively higher noise in the tactile modality (or other modalities in other examples) can result in a relatively greater risk of false positives. Detecting the occurrence of the low-noise scenario 910 can be based on performing operation 640, which can include determining that a stillness of a recipient of the medical device passes a stillness threshold.

Where the sensor modality is an audio modality, the operation 912 can include operation 914, operation 916, or operation 918. Operation 914 can include determining that a volume of a sound environment satisfies a low-noise threshold. Sound in the sound environment is provided by, for example, a television, radio, sound system, people talking, a fan operating, wind noise, or other sources. For instance, a volume of the sound environment is determined to be below a threshold. Operation 916 can include determining based on a classifier of the medical device 110 that the input was received in a quiet environment. For example, where the medical device 110 is being controlled with audio commands 10, the low noise scenario occurs in a quiet sound environment as determined by a classifier of the medical device 110. Because the sound environment is quiet, the medical device 110 (or whichever device is performing the operation) can determine that the audible command 10 or other detected sounds were likely interpreted correctly. Operation 918 can include determining absence of a conversation. Absence of conversation (e.g., detecting a lack of turn taking) can be considered a low false positive probability scenario. Whereas the presence of a conversation can be considered a relatively higher false positive probability scenario. For instance, the conversation may include words or phrases that happen to correspond to commands but are not intended to be used as commands, which can lead to false positives. Certain kinds of devices (e.g., totally implanted devices) can be configured to detect own-voice events that readily permit the determination of whether a conversation is occurring.

The consistent-context scenario 920 can be a scenario 900 whereby received commands 10 are what would be expected for a given context. The medical device 110 can store a data structure of contexts and commands predetermined to be consistent with such contexts. To determine whether a command 10 is consistent with the context can include comparing a current context and command 10 with an associated entry in the data structure. As an example, the command 10 is to reduce a volume/level and the volume/level is high relative to the current ambient sound level as detected based on data from one or more of the sensors 112. As another example, the command 10 is to switch programs in the context of a recently-changed sound environment (e.g., going from a windy environment to a non-windy environment where music is playing as based on a scene classifier of the medical device 110). As yet another example, the command 10 is to reduce a volume or power off in the context of a very high or indeterminable output, which may indicate a potential malfunction of the medical device 110. To detect the occurrence of the consistent-context scenario 920 can include the performance of operation 620. In an example, the occurrence of the consistent-context scenario 920 is detected based on comparing the command 10 with a context in which the medical device 110 is operating.

The consistent-behavior scenario 930 can be a scenario 900 in which the command 10 is consistent with past behavior. The medical device 110 can store a data structure of commands that the user typically provides. To determine whether a command 10 is consistent with the context can include comparing a command 10 with an associated entry in the data structure. In some examples, the data structure includes contexts and associate contexts with commands that the medical device 110 typically receives during those contexts. For example, based on data logging, a command 10 is determined to be typical for the recipient given a context and therefore a consistent-behavior scenario 930 can be determined to exist. For instance, the command 10 is determined to be typical for the recipient when entering a certain physical location, connecting to a certain device, connecting to or being proximate a certain wireless network (e.g., a WI-FI network, such as may be determined based on an SSID of the network), or entering a certain audio environment. To detect the occurrence of the consistent-behavior scenario 930 can be based on operation 630. For example, detecting the occurrence of the consistent-behavior scenario includes comparing the command 10 with a past behavior of the medical device 110.

The activity scenario 940 can be a scenario 900 based on the recipient's activity. For example, the recipient is determined to be concentrating on other activities, using both hands, or is in need of reduced complexity. In these situations, the recipient may have an increased tolerance for false positives. For instance, the recipient may be driving (e.g., as determined by connection to an automobile audio system), texting (e.g., as determined and communicated to the prosthesis by a mobile device in use for texting), watching video content (e.g., as determined by use of wireless accessories), riding a bicycle (e.g., as determined by an accelerometer and digital/wireless maps), or showering (e.g., as determined by an implantable microphone). Such scenarios 900 do not typically represent the majority of the recipient's time while using the medical device 110. They are instead exceptions to typical behavior for most recipients. Entering wake commands for some such scenarios 900 could be dangerous. For instance, while driving or riding a bike, a recipient may not want to have their attention diverted to satisfy a verification process. In other such scenarios, the recipient may not be able to do more. For instance, while showering or texting, the recipient might not have sufficient physical or mental dexterity to engage in such activities while satisfying a verification process. In another example, the recipient is ill or has diminished capacity (e.g., as determined by implanted temperature sensors). Detecting the occurrence of the activity scenario 940 can include determining that an activity level of a recipient satisfies a threshold, such as is described in operation 640. In addition or instead, detecting the occurrence of the activity scenario 940 can include the performance of operation 942. Operation 942 includes comparing the command 10 with a predicted activity level of a recipient of the medical device 110. In an example, the activity scenario 940 is determined responsive to the medical device 110 predicting that a recipient is in a high-activity scenario 940 based on determining that a recipient of the medical device 110 is: driving based on a connection to an automobile audio system, communicating using a mobile device, consuming media content based on use of wireless accessories, engaging in physical activity based on output of an accelerometer, showering based on output of a microphone, or based on output of a temperature sensor. In some examples, the occurrence of the activity scenario is determined based on operation 640, which includes determining whether an activity level of the recipient satisfies a threshold.

### Example Devices

As previously described, the technology disclosed herein can be applied in any of a variety of circumstances and with a variety of different devices. Example devices that can benefit from technology disclosed herein are described in more detail in FIGS. 10-13, below. For example, the techniques described herein control medical devices, such as an implantable stimulation system as described in FIG. 10, a cochlear implant as described in FIG. 11, a bone conduction device as described in FIG. 12, or a retinal prosthesis as described in FIG. 13. The technology can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

### Example Device—Implantable Stimulator System

FIG. 10 is a functional block diagram of an implantable stimulator system 1000 that can benefit from the technologies described herein. The implantable stimulator system 1000 includes the wearable device 1010 acting as an external processor device and an implantable device 1050 acting as an implanted stimulator device. The implantable stimulator system 1000 and its components can correspond to the medical device 110. In examples, the implantable device 1050 is an implantable stimulator device configured to be implanted beneath a recipient's tissue (e.g., skin). In examples, the implantable device 1050 includes a biocompatible implantable housing 1002. Here, the wearable device 1010 is configured to transcutaneously couple with the implantable device 1050 via a wireless connection to provide additional functionality to the implantable device 1050.

In the illustrated example, the wearable device 1010 includes one or more sensors 112, a processor 114, a transceiver 118, and a power source 1048. The one or more sensors 112 can be units configured to produce data based on sensed activities. In an example where the stimulation system 1000 is an auditory prosthesis system, the one or more sensors 112 include sound input sensors, such as a microphone, an electrical input for an FM hearing system, other components for receiving sound input, or combinations thereof. Where the stimulation system 1000 is a visual prosthesis system, the one or more sensors 112 can include one or more cameras or other visual sensors. Where the stimulation system 1000 is a cardiac stimulator, the one or more sensors 112 can include cardiac monitors. The processor 114 can be a component (e.g., a central processing unit) configured to control stimulation provided by the implantable device 1050. The stimulation can be controlled based on data from the sensor 112, a stimulation schedule, or other data. Where the stimulation system 1000 is an auditory prosthesis, the processor 114 can be configured to convert sound signals received from the sensor(s) 112 (e.g., acting as a sound input unit) into signals 1051. The transceiver 118 is configured to send the signals 1051 in the form of power signals, data signals, combinations thereof (e.g., by interleaving the signals), or other signals. The transceiver 118 can also be configured to receive power or data. Stimulation signals can be generated by the processor 114 and transmitted, using the transceiver 118, to the implantable device 1050 for use in providing stimulation.

In the illustrated example, the implantable device 1050 includes a transceiver 118, a power source 1048, a coil 1056, and a medical instrument 111 that includes an electronics module 1010 and a stimulator assembly 1030. The implantable device 1050 further includes a hermetically sealed, biocompatible housing enclosing one or more of the components.

The electronics module 1010 can include one or more other components to provide medical device functionality. In many examples, the electronics module 1010 includes one or more components for receiving a signal and converting the signal into the stimulation signal 1015. The electronics module 1010 can further include a stimulator unit. The electronics module 1010 can generate or control delivery of the stimulation signals 1015 to the stimulator assembly 1030. In examples, the electronics module 1010 includes one or more processors (e.g., central processing units or microcontrollers) coupled to memory components (e.g., flash memory) storing instructions that when executed cause performance of an operation. In examples, the electronics module 1010 generates and monitors parameters associated with generating and delivering the stimulus (e.g., output voltage, output current, or line impedance). In examples, the electronics module 1010 generates a telemetry signal (e.g., a data signal) that includes telemetry data. The electronics module 1010 can send the telemetry signal to the wearable device 1010 or store the telemetry signal in memory for later use or retrieval.

The stimulator assembly 1030 can be a component configured to provide stimulation to target tissue. In the illustrated example, the stimulator assembly 1030 is an electrode assembly that includes an array of electrode contacts disposed on a lead. The lead can be disposed proximate tissue to be stimulated. Where the system 1000 is a cochlear implant system, the stimulator assembly 1030 can be inserted into the recipient's cochlea. The stimulator assembly 1030 can be configured to deliver stimulation signals 1015 (e.g., electrical stimulation signals) generated by the electronics module 1010 to the cochlea to cause the recipient to experience a hearing percept. In other examples, the stimulator assembly 1030 is a vibratory actuator disposed inside or outside of a housing of the implantable device 1050 and configured to generate vibrations. The vibratory actuator receives the stimulation signals 1015 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 118 can be components configured to transcutaneously receive and/or transmit a signal 1051 (e.g., a power signal and/or a data signal). The transceiver 118 can be a collection of one or more components that form part of a transcutaneous energy or data transfer system to transfer the signal 1051 between the wearable device 1010 and the implantable device 1050. Various types of signal transfer, such as electromagnetic, capacitive, and inductive transfer, can be used to usably receive or transmit the signal 1051. The transceiver 118 can include or be electrically connected to the coil 1056.

The coils 1056 can be components configured to receive or transmit a signal 1051, typically via an inductive arrangement formed by multiple turns of wire. In examples, in addition to or instead of a coil, other arrangements are used, such as an antenna or capacitive plates. The magnets can be used to align respective coils 1056 of the wearable device 1010 and the implantable device 1050. For example, the coil 1056 of the implantable device 1050 is disposed in relation to (e.g., in a coaxial relationship) with an implantable magnet set to facilitate orienting the coil 1056 in relation to the coil 1056 of the wearable device 1010 via the force of a magnetic connection. The coil 1056 of the wearable device 1010 can be disposed in relation to (e.g., in a coaxial relationship) with a magnet set.

The power source 1048 can be one or more components configured to provide operational power to other components. The power source 1048 can be or include one or more rechargeable batteries. Power for the batteries can be received from a source and stored in the battery. The power can then be distributed to the other components of the implantable device 1050 as needed for operation.

As should be appreciated, while particular components are described in conjunction with FIG. 10, technology disclosed herein can be applied in any of a variety of circumstances. The above discussion is not meant to suggest that the disclosed techniques are only suitable for implementation within systems akin to that illustrated in and described with respect to FIG. 10. In general, additional configurations can be used to practice the methods and systems herein and/or some aspects described can be excluded without departing from the methods and systems disclosed herein.

### Example Device—Cochlear Implant

FIG. 11 illustrates an example cochlear implant system 1110 that can benefit from use of the technologies disclosed herein. For example, the cochlear implant system 1110 corresponds to the medical device 110 and be controlled using one or more aspects of disclosed technology. The cochlear implant system 1110 includes an implantable component 1144 typically having an internal receiver/transceiver unit 1132, a stimulator unit 1120, and an elongate lead 1118. The internal receiver/transceiver unit 1132 permits the cochlear implant system 1110 to receive signals from and/or transmit signals to an external device 1150. The external device 1150 can be a button sound processor worn on the head that includes a receiver/transceiver coil 1130 and sound processing components. Alternatively, the external device 1150 can be just a transmitter/transceiver coil in communication with a behind-the-ear device that includes the sound processing components and microphone.

The implantable component 1144 includes an internal coil 1136, and preferably, an implanted magnet fixed relative to the internal coil 1136. The magnet can be embedded in a pliable silicone or other biocompatible encapsulant, along with the internal coil 1136. Signals sent generally correspond to external sound 1113. The internal receiver/transceiver unit 1132 and the stimulator unit 1120 are hermetically sealed within a biocompatible housing, sometimes collectively referred to as a stimulator/receiver unit. Included magnets can facilitate the operational alignment of an external coil 1130 and the internal coil 1136 (e.g., via a magnetic connection), enabling the internal coil 1136 to receive power and stimulation data from the external coil 1130. The external coil 1130 is contained within an external portion. The elongate lead 1118 has a proximal end connected to the stimulator unit 1120, and a distal end 1146 implanted in a cochlea 1140 of the recipient. The elongate lead 1118 extends from stimulator unit 1120 to the cochlea 1140 through a mastoid bone 1119 of the recipient. The elongate lead 1118 is used to provide electrical stimulation to the cochlea 1140 based on the stimulation data. The stimulation data can be created based on the external sound 1113 using the sound processing components and based on sensory prosthesis settings.

In certain examples, the external coil 1130 transmits electrical signals (e.g., power and stimulation data) to the internal coil 1136 via a radio frequency (RF) link. The internal coil 1136 is typically a wire antenna coil having multiple turns of electrically insulated single-strand or multi-strand platinum or gold wire. The electrical insulation of the internal coil 1136 can be provided by a flexible silicone molding. Various types of energy transfer, such as infrared (IR), electromagnetic, capacitive and inductive transfer, can be used to transfer the power and/or data from external device to cochlear implant. While the above description has described internal and external coils being formed from insulated wire, in many cases, the internal and/or external coils can be implemented via electrically conductive traces.

### Example Device—Percutaneous Bone Conduction Device

FIG. 12 is a view of an example of a percutaneous bone conduction device 1200 that can benefit from use of the technologies disclosed herein. For example, the percutaneous bone conduction device 1200 corresponds to the medical device 110 and be controlled using one or more aspects of disclosed technology. The bone conduction device 1200 is positioned behind an outer ear 1201 of a recipient of the device. The bone conduction device 1200 includes a sound input element 1226 to receive sound signals 1207. The sound input element 1226 can be a microphone, telecoil or similar. In the present example, the sound input element 1226 is located, for example, on or in the bone conduction device 1200, or on a cable extending from the bone conduction device 1200. Also, the bone conduction device 1200 comprises a sound processor (not shown), a vibrating electromagnetic actuator and/or various other operational components.

More particularly, the sound input element 1226 converts received sound signals into electrical signals. These electrical signals are processed by the sound processor. The sound processor generates control signals that cause the actuator to vibrate. In other words, the actuator converts the electrical signals into mechanical force to impart vibrations to a skull bone 1236 of the recipient. The conversion of the electrical signals into mechanical force can be controlled by input received from a user.

The bone conduction device 1200 further includes a coupling apparatus 1240 to attach the bone conduction device 1200 to the recipient. In the illustrated example, the coupling apparatus 1240 is attached to an anchor system (not shown) implanted in the recipient. An exemplary anchor system (also referred to as a fixation system) includes a percutaneous abutment fixed to the skull bone 1236. The abutment extends from the skull bone 1236 through muscle 1234, fat 1228 and skin 1232 so that the coupling apparatus 1240 can be attached thereto. Such a percutaneous abutment provides an attachment location for the coupling apparatus 1240 that facilitates efficient transmission of mechanical force.

### Example Device—Retinal Prosthesis

FIG. 13 illustrates a retinal prosthesis system 1301 that comprises an external device 1310, a retinal prosthesis 1300 and a mobile computing device 1303. The retinal prosthesis system 1301 can correspond to the medical device 110 and be controlled using one or more aspects of disclosed technology. The retinal prosthesis 1300 comprises a processing module 1325 and a retinal prosthesis sensor-stimulator 1390 is positioned proximate the retina 1391 of a recipient. The external device 1310 and the processing module 1325 can both include transmission coils 1356 aligned via respective magnet sets. Signals 1351 can be transmitted using the coils 1356.

In an example, sensory inputs (e.g., photons entering the eye) are absorbed by a microelectronic array of the sensor-stimulator 1390 that is hybridized to a glass piece 1392 including, for example, an embedded array of microwires. The glass can have a curved surface that conforms to the inner radius of the retina. The sensor-stimulator 1390 can include a microelectronic imaging device that can be made of thin silicon containing integrated circuitry that convert the incident photons to an electronic charge.

The processing module 1325 includes an image processor 1323 that is in signal communication with the sensor-stimulator 1390 via, for example, a lead 1388 which extends through surgical incision 1389 formed in the eye wall. In other examples, processing module 1325 is in wireless communication with the sensor-stimulator 1390. The image processor 1323 processes the input into the sensor-stimulator 1390, and provides control signals back to the sensor-stimulator 1390 so the device can provide an output to the optic nerve. That said, in an alternate example, the processing is executed by a component proximate to, or integrated with, the sensor-stimulator 1390. The electric charge resulting from the conversion of the incident photons is converted to a proportional amount of electronic current which is input to a nearby retinal cell layer. The cells fire and a signal is sent to the optic nerve, thus inducing a sight perception.

The processing module 1325 can be implanted in the recipient and function by communicating with the external device 1310, such as a behind-the-ear unit, a pair of eyeglasses, etc. The external device 1310 can include an external light/image capture device (e.g., located in/on a behind-the-ear device or a pair of glasses, etc.), while, as noted above, in some examples, the sensor-stimulator 1390 captures light/images, which sensor-stimulator is implanted in the recipient.

Similar to the above examples, the retinal prosthesis system 1301 may be used in spatial regions that have at least one controllable network connected device associated therewith (e.g., located therein). As such, the processing module 1325 includes a performance monitoring engine 1327 that is configured to obtain data relating to a “sensory outcome” or “sensory performance” of the recipient of the retinal prosthesis 1300 in the spatial region. As used herein, a “sensory outcome” or “sensory performance” of the recipient of a sensory prosthesis, such as retinal prosthesis 1300, is an estimate or measure of how effectively stimulation signals delivered to the recipient represent sensor input captured from the ambient environment.

Data representing the performance of the retinal prosthesis 1300 in the spatial region is provided to the mobile computing device 1303 and analyzed by a network connected device assessment engine 1362 in view of the operational capabilities of the at least one controllable network connected device associated with the spatial region. For example, the network connected device assessment engine 1362 may determine one or more effects of the controllable network connected device on the sensory outcome of the recipient within the spatial region. The network connected device assessment engine 1362 is configured to determine one or more operational changes to the at least one controllable network connected device that are estimated to improve the sensory outcome of the recipient within the spatial region and, accordingly, initiate the one or more operational changes to the at least one controllable network connected device.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A method comprising:
monitoring sensor data for a pre-defined command;
determining whether to require a verification process; and
based on the determining, controlling a medical device based on the pre-defined command.

2. The method of claim 1, wherein determining whether to require the verification process includes:
determining whether a false positive command probability passes a false positive command probability threshold.

3. The method of claim 1, wherein determining whether to require the verification process includes:
determining whether the pre-defined command is consistent with a current context.

4. The method of any of claim 1, wherein determining whether to require the verification process includes:
determining whether the pre-defined command is consistent with data regarding past behavior.

5. The method of any of claim 1, wherein determining whether to require the verification process includes:
determining whether an activity level of a recipient of the medical device passes a threshold.

6. The method of claim 1, wherein monitoring sensor data for the pre-defined command includes obtaining first sensor data from a first sensor, and wherein the method further comprises:
obtaining second sensor data from a second sensor,
wherein determining whether to require a verification process is based on the second sensor data.

7. The method of claim 6, wherein determining whether to require the verification process is further based on the first sensor data.

8. The method of claim 1, wherein when the verification process is determined to be required, the method further comprises:
performing the verification process by monitoring for a predetermined wake input,
wherein the monitoring the sensor data for the pre-defined command is responsive to detecting the predetermined wake input.

9. The method of claim 1, wherein when the verification process is determined to be required, the method further comprises:
performing the verification process by requesting confirmation that the pre-defined command is to be performed; and
controlling the medical device based on the pre-defined command responsive to receiving the confirmation that the pre-defined command is to be performed.

10. The method of claim 1, wherein monitoring the sensor data for the pre-defined command includes monitoring for a voice command or a tap command, and the method further includes:
performing the verification process by verifying via a device proximate to the medical device, wherein the medical device is at least one of a sensory prosthesis, implantable stimulator, or a drug pump.

11. A system comprising:
one or more processors configured to:
obtain input defining a command;
control a medical device based on the command;
selectively perform a verification process prior to controlling the medical device based on the input; and
bypass the verification process responsive to detecting an occurrence of one or more scenarios.

12. The system of claim 11, wherein the one or more scenarios include a low-noise scenario and wherein to detect the occurrence of the low-noise scenario, the one or more processors are configured to determine activity within a modality of the input defining the command.

13. The system of claim 11, wherein the one or more scenarios include a consistent-context scenario, and wherein to detect the occurrence of the consistent-context scenario includes comparing the command with a context in which the medical device is operating.

14. The system of claim 11, wherein the one or more scenarios include a consistent-behavior scenario, and wherein to detect the occurrence of the consistent-behavior scenario, the one or more processors are configured to compare the command with a past behavior.

15. The system of claim 11, wherein the one or more scenarios include an activity scenario, and wherein to detect the occurrence of the activity scenario, the one or more processors are configured to compare the command with a predicted activity level of a recipient of the medical device.

16. The system of claim 11, further comprising:
a first sensor configured to obtain the input; and
a second sensor,
wherein to perform the verification process, the one or more processors are configured to obtain data from the second sensor.

17. The system of claim 11, wherein the one or more processors are configured to selectively bypass the verification process responsive to detecting the occurrence of at least two or more of the scenarios contemporaneous with the input.

18. The system of any of claim 11, wherein the one or more processors are configured to, responsive to the input failing the verification process, prevent controlling a function of the medical device based on the input.

19. The system of claim 11, further comprising:
the medical device; and
a control device separate from the medical device comprising the one or more processors,
wherein to control the medical device based on the command, the one or more processors are configured to transmit a message from the control device to the medical device.

20. (canceled)

21. An apparatus comprising:
a stimulator;
a sensor; and
one or more processors configured to:
stimulate a system of a recipient using the stimulator;
receive an input from the sensor;
determine whether the input passes a verification process based on the input including a wake input;
detect occurrence of at least one bypass scenario; and
control stimulation of the system of the recipient based on the input responsive to either:
the input including a command proximate the wake input; or
the at least one bypass scenario occurring.

22. The apparatus of claim 21, wherein the at least one bypass scenario includes a low-noise scenario, and wherein to detect the occurrence of the low-noise scenario, the one or more processors are configured to determine that a volume of a sound environment satisfies a low-noise threshold.

23. The apparatus of claim 21, wherein the at least one bypass scenario includes a high-activity scenario, and wherein to detect the occurrence of the high-activity scenario, the one or more processors are configured to determine that an activity level of the recipient satisfies a high-activity threshold.

24. The apparatus of claim 21, further comprising:
an implantable, biocompatible housing comprising the stimulator, the sensor, and the one or more processors.

25. The apparatus of claim 21, wherein to control the stimulation of the system of the recipient based on the input responsive to the input including a command proximate the wake input includes the command being proximate the wake input and before the wake input.

26. A computer-readable medium having instructions stored thereon that, when executed by one or more processors, cause the one or more processors to:
obtain an input comprising a command;
determine whether a bypass scenario occurred;
responsive to failing to determine that the bypass scenario occurred, require verification prior to executing the command; and
control a medical device based the command.

27. The computer-readable medium of claim 26, further comprising instructions stored thereon that, when executed by one or more processors, cause the one or more processors to:
determine that the bypass scenario occurred responsive to at least one of:
determining based on a classifier of the medical device that the input was received in a quiet environment;
determining absence of a conversation; or
determining that a stillness of a recipient of the medical device passes a stillness threshold.

28. The computer-readable medium of claim 26, further comprising instructions stored thereon that, when executed by one or more processors, cause the one or more processors to:
determine that the bypass scenario occurred responsive to at least one of:
determining that the command is consistent with a current context by being a command to decrease a volume level of the medical device and that the input was received in a noisy environment;
determining that the command is consistent with a current context by being a command to increase a volume level of the medical device and that the input was received in a quiet environment;
determining that the command is consistent with a current context by being a command to change an operating mode of the medical device and a sound environment of the medical device changed within a threshold amount of time; or
determining that the command is consistent with a current context by being a command to deactivate the medical device and an output of the medical device is higher than an output threshold.

29. The computer-readable medium of claim 26, further comprising instructions stored thereon that, when executed by one or more processors, cause the one or more processors to:
determine that the bypass scenario occurred responsive to:
determining that the command is consistent with commands previously provided by a recipient when at a certain physical location, connected to a certain device, or in a certain audio environment.

30. The computer-readable medium of claim 26, further comprising instructions stored thereon that, when executed by one or more processors, cause the one or more processors to:
determine that the bypass scenario occurred responsive to determining that a recipient is in a high-activity scenario based on determining that a recipient of the medical device is at least one of:
driving based on a connection to an automobile audio system;
communicating using a mobile device;
consuming media content based on use of wireless accessories;
engaging in physical activity based on output of an accelerometer;
showering based on output of a microphone; or
based on output of a temperature sensor.
